(19) United States
(12) Patent Application Publication
(10) Pub. No.: US 2025/0062014 A1
(43) Pub. Date: Feb. 20, 2025

(54) DYNAMIC MEDICAL SUPPLY PROCUREMENT

(71) Applicant: KONINKLIJKE PHILIPS N.V., EINDHOVEN (NL)

(72) Inventors: JIN LIU, MALDEN, MA (US); LUCAS DE MELO OLIVEIRA, WILMINGTON, MA (US)

(21) Appl. No.: 18/719,302

(22) PCT Filed: Dec. 11, 2022
(86) PCT No.: PCT/EP2022/085269

§ 371 Date: Jun. 13, 2024

Related U.S. Application Data
(60) Provisional application No. 63/291,581, filed on Dec. 20, 2021.

(51) Int. Cl. G16H 40/20; G06F 40/40; G16H 10/60; G16H 70/20

(52) U.S. Cl. CPC G16H 40/20; G06F 40/40; G16H 10/60; G16H 70/20

(57) ABSTRACT

A method for dynamically obtaining a supply includes storing, in a main memory (115), identification of supplies used during a medical procedure: monitoring information from the medical procedure during the medical procedure: predicting, by a processor (152) executing instructions and based on monitoring the information, whether a supply that is not present should be obtained during the medical procedure, and obtaining the supply that is not present during the medical procedure based on predicting that the supply that is not present should be obtained.

### BACKGROUND

The cost of cardiovascular diseases to the United States economy may have been more than 500 billion dollars in 2017, and is projected by some to reach 1.1 trillion dollars by 2035. Thirty percent or so of these costs may be due to waste and inefficiency in healthcare, such as due to inefficient supply chains and waste in surgeries. The avoidable cost of cardiovascular diseases is seen in clinical workflow such as in catheterization laboratories, where consumable devices account for a considerable cost of procedures.

The cost of healthcare in the United States has led to pressure to increase operational efficiency and improve inventory distribution, while maintaining or improving clinical outcomes. Usage of a supply may be difficult to predict ahead of time. Moreover, if a supply at one wing and/or floor of a hospital is running short, staff may need to obtain the supply from a different wing and/or floor, or even a different hospital, resulting in longer wait times for patients even when the patients are in critical condition or in surgery. Additionally, usage of a supply may vary by preference and/or ability of a medical professional who is in the position to use the supply. Improved clinical outcomes may depend on an ability to customize supply procurement according to staff preferences and/or abilities, and patient conditions, to achieve improved clinical outcomes. In addition, the price of a supply may vary over time and may be different than alternatives that can be used with equally acceptable clinical outcomes.

### SUMMARY

According to an aspect of the present disclosure, a method for dynamically obtaining a supply may include storing, in a main memory, identification of supplies used during a medical procedure: monitoring information from the medical procedure during the medical procedure; predicting, by a processor executing instructions and based on monitoring the information, whether a supply that is not present should be obtained during the medical procedure, and obtaining the supply that is not present during the medical procedure based on predicting that the supply that is not present should be obtained.

According to another aspect of the present disclosure, a system for dynamically obtaining a supply includes a central computer and a main memory. The central computer comprises a first memory that stores first instructions and a first processor that executes the first instructions. The main memory stores identification of supplies used during a medical procedure. When executed by the first memory, the first instructions cause the central computer to: monitor information from the medical procedure during the medical procedure: predict, based on monitoring the information, whether a supply that is not present should be obtained during the medical procedure, and obtain the supply that is not present during the medical procedure based on predicting that the supply that is not present should be obtained.

According to another aspect of the present disclosure, a controller includes a memory and a processor. The memory stores instructions. The processor executes the instructions. When executed by the processor, the instructions cause the controller to: obtain identification of supplies used during the medical procedure: monitor information from the medical procedure during the medical procedure: predict, based on monitoring the information, whether a supply that is not present should be obtained during the medical procedure, and obtain the supply that is not present during the medical procedure based on predicting that the supply that is not present should be obtained.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for dynamic medical supply procurement, in accordance with a representative embodiment.

FIG. 1B illustrates another system for dynamic medical supply procurement, in accordance with a representative embodiment.

FIG. 1C illustrates another system for dynamic medical supply procurement, in accordance with a representative embodiment.

FIG. 1D illustrates a controller for dynamic medical supply procurement, in accordance with a representative embodiment.

FIG. 1E illustrates a system overview for dynamic medical supply procurement, in accordance with a representative embodiment.

FIG. 2 illustrates a method for dynamic medical supply procurement, in accordance with a representative embodiment.

FIG. 3A illustrates a method for dynamic medical supply procurement, in accordance with a representative embodiment.

FIG. 3B illustrates insight generation for dynamic medical supply procurement, in accordance with a representative embodiment.

FIG. 4 illustrates a computer system, on which a method for dynamic medical supply procurement is implemented, in accordance with another representative embodiment.

FIG. 5A illustrates a user interface for a manager/provider computer for dynamic medical supply procurement, in accordance with a representative embodiment.

FIG. 5B illustrates another user interface for a manager/provider computer for dynamic medical supply procurement, in accordance with a representative embodiment

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, inventory management systems may be supplemented and enhanced by generating and leveraging insights on supplies used in clinical procedures by different staff in order to provide real-time predictions and suggestions. The ability to provide real-time predictions and suggestions may benefit from information derived from clinical procedures, including clinical information, operational information and/or workflow information. As a result, healthcare providers may be provided a comprehensive overview of supply usage, staff preferences, adherence to guidelines, specific trends and opportunities to improve performance.

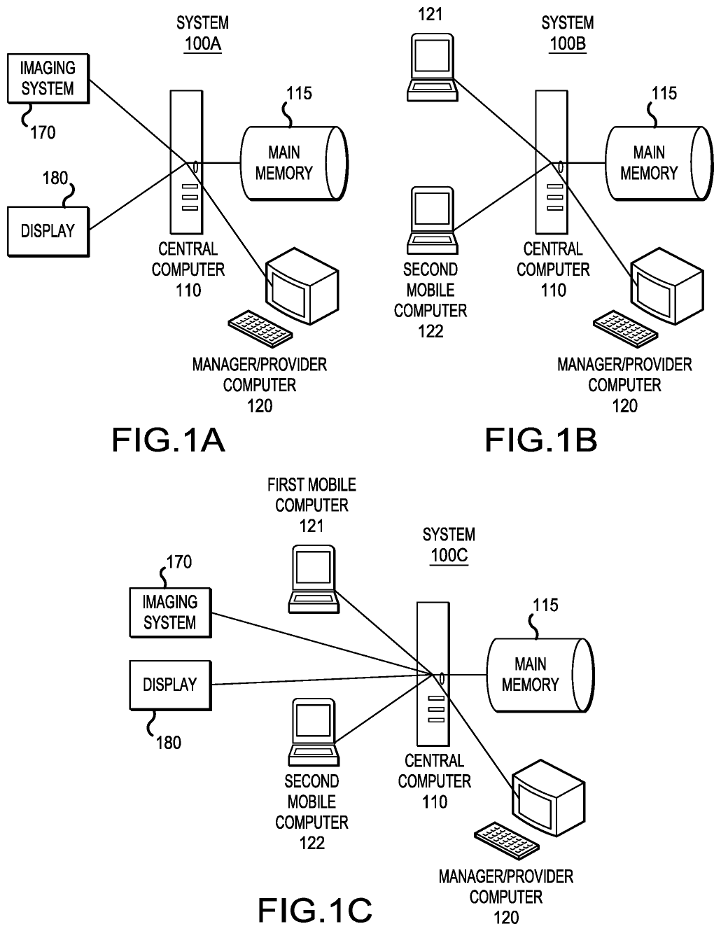

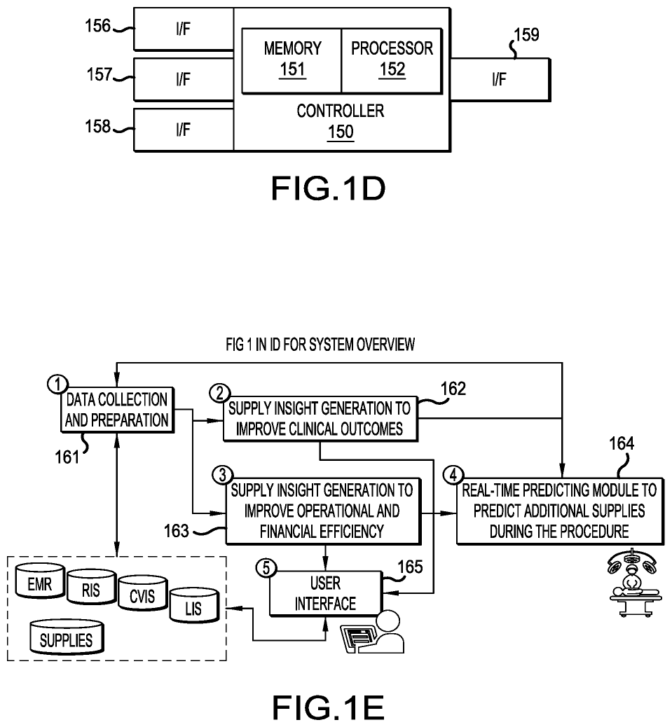

FIG. 1A illustrates a system 100A for dynamic medical supply procurement, in accordance with a representative embodiment.

The system 100A in FIG. 1A is a system for dynamic medical supply procurement and includes components that may be provided together or that may be distributed. The system 100A includes a central computer 110, a main memory 115, a manager/provider computer 120, an imaging system 170 and a display 180.

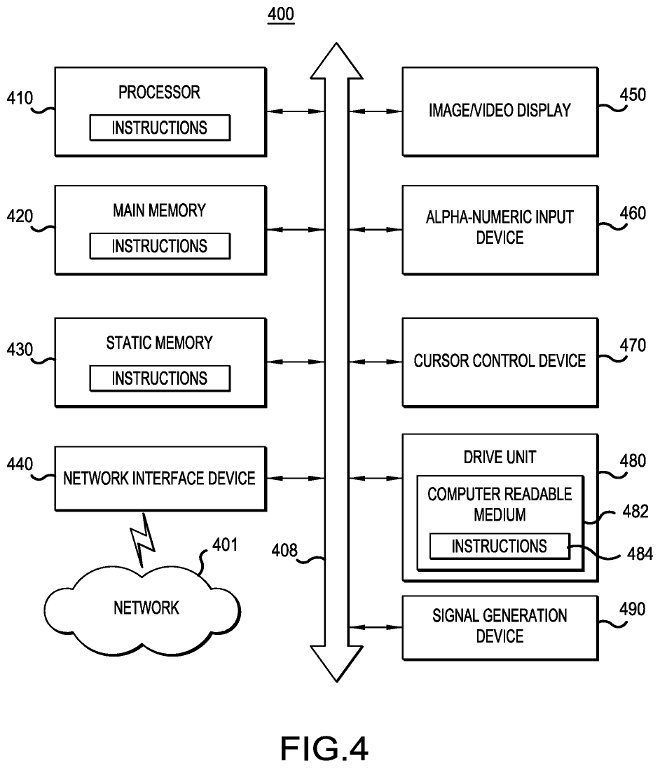

The central computer 110 may be a server computer, a desktop computer, or another type of computer. The central computer 110 includes a memory that stores instructions and a processor that executes the instructions. The central computer 110 may comprise a single computer, or a set of multiple coordinated computers such as in a data center. A computer that may be used to implement the central computer is depicted in FIG. 4, though a central computer 110 may include more or fewer elements than depicted in FIG. 4. The central computer 110 may perform some or even most aspects of methods described herein.

The main memory 115 may comprise a non-volatile memory such as a flash memory. The main memory 115 may store one or more types of information from previous medical procedures which were previously performed. The main memory 115 may store historical information for one or more staff members, such as previous medical procedures they were involved in and types of supplies they used during the previous medical procedures. The main memory 115 may comprise a single, integrated memory, or a set of multiple coordinated memories.

The manager/provider computer 120 may be a desktop computer, a laptop computer, or another type of computer. The manager/provider computer 120 includes a memory that stores instructions and a processor that executes the instructions. A computer that may be used to implement the manager/provider computer 120 is depicted in FIG. 4, though a manager/provider computer 120 may include more or fewer elements than depicted in FIG. 4. The manager/provider computer 120 is representative of computers used by managers and providers to input and retrieve data from the main memory 115. For example, the manager/provider computer 120 may be used to input user-defined rules for the central computer 110 to use when predicting whether a supply that is not present during a medical procedure should be obtained during the medical procedure.

The imaging system 170 may be a medical imaging system, such as an ultrasound system or an X-ray system. The imaging system 170 may also comprise a set of multiple different medical imaging systems, such as an ultrasound system and an X-ray system. The imaging system 170 may generate medical images during a medical procedure and provide the medical images and/or information derived from the medical images to the central computer 110. The medical images from the imaging system 170 may be analyzed to predict when a supply that is not present should be obtained. The analysis of the medical images from the imaging system 170 may involve the quality of the medical images, such as whether the imaging system 170 is producing medical images of an acceptable quality. The analysis of the medical images from the imaging system 170 may also or alternatively involve medical analysis, such as whether the medical images show anatomical characteristics consistent with expectations for the medical procedure.

The display 180 may be local to the imaging system 170, local to the central computer 110 and/or local to the manager/provider computer 120. The display 180 may also comprise multiple different displays. The display 180 may be connected to the central computer 110 via a local wired interface such as an Ethernet cable or via a local wireless interface such as a Wi-Fi connection. The display 180 may be interfaced with other user input devices by which users may input instructions, including mouses, keyboards, thumbwheels and so on.

The display 180 may be a monitor such as a computer monitor, an augmented reality display, a television, an electronic whiteboard, or another screen configured to display electronic imagery. The display 180 may also include one or more input interface(s) that may connect other elements or components to the central computer 110, as well as an interactive touch screen configured to display prompts to users and collect touch input from users.

FIG. 1B illustrates another system for dynamic medical supply procurement, in accordance with a representative embodiment.

The system 100B in FIG. 1B is a system for dynamic medical supply procurement and includes components that may be provided together or that may be distributed. The system 100B includes a central computer 110, a main memory 115, a manager/provider computer 120, a first mobile computer 121 and a second mobile computer 122.

The elements of FIG. 1B which are the same as in FIG. 1A are not reintroduced for the sake of brevity. The first mobile computer 121 includes a memory that stores instructions and a processor that executes the instructions. The second mobile computer 122 includes a memory that stores instructions and a processor that executes the instructions. The first mobile computer 121 and the second mobile computer 122 may each be representative of a communication device such as a mobile tablet computers used by staff such as nurses and technicians during medical procedures. The first mobile computer 121 and the second mobile computer 122 may be used to input structured and formatted data and instructions and/or unstructured and free-form data and instructions. As an example, the staff may input clinical information to the first mobile computer 121 or the second mobile computer 122 during medical procedures. Clinical information may refer to clinical concepts and patient demographic information As another example, the staff may input operational information to the first mobile computer 121 or the second mobile computer 122 during medical procedures. Operational information may refer to information relating to the operation of a given hospital or department(s) in a hospital. When the data and/or instructions are free-form, natural language processing may be applied to the data and/or instructions in order to interpret the information from the medical procedure.

FIG. 1C illustrates another system for dynamic medical supply procurement, in accordance with a representative embodiment.

The system 100C in FIG. 1C is a system for dynamic medical supply procurement and includes components that may be provided together or that may be distributed. The system 100C includes a central computer 110, a main memory 115, a manager/provider computer 120, a first mobile computer 121 a second mobile computer 122, an imaging system 170 and a display 180.

The elements of FIG. 1C which are the same as in FIG. 1A and/or FIG. 1B are not reintroduced for the sake of brevity. FIG. 1C reinforces a concept that a system described herein may include more elements, less elements, and different elements than are shown in FIG. 1A, FIG. 1B and/or FIG. 1C. Functionality of methods described herein is primarily attributed to the central computer 110, but may also invoke functionality of the other elements in the systems of FIG. 1A, FIG. 1B and/or FIG. 1C, along with other elements which are not shown.

The central computer 110 may analyze clinical information input to the first mobile computer 121 or the second mobile computer 122 and received from the first mobile computer 121 or the second mobile computer 122 in order to determine compliance with clinical guidelines during the medical procedure. Also, or alternatively, the central computer 110 may analyze operational information input to the first mobile computer 121 or the second mobile computer 122 and received from the first mobile computer 121 or the second mobile computer 122 in order to determine usage of supplies during the medical procedure.

In any of FIG. 1A, FIG. 1B and/or FIG. 1C, the information received by the central computer 110 may be obtained from a plurality of different sources over an electronic communication network. For example, the central computer 110 may be connected to a broadband cable such as an Ethernet cable, and may receive information from a medical procedure from any or all of the imaging system 170, the first mobile computer 121, the second mobile computer 122 or a variety of other types of electronic devices and systems which may be present at the medical procedure. The central computer 110 may process information received from one or a plurality of sources in order to monitor the medical procedure and predict whether a supply which is not present should be obtained.

FIG. 1D illustrates controller 150 for dynamic medical supply procurement, in accordance with a representative embodiment.

The controller 150 includes a memory 151, a processor 152, a first interface 156, a second interface 157, a third interface 158, and a fourth interface 159. The memory 151 stores instructions. The processor 152 executes the instructions.

A computer that may be used to implement the controller 150 may be the central computer 110, the manager/provider computer 120, the first mobile computer 121 and/or the second mobile computer 122. A computer which may be used to implement the controller 150 is depicted in FIG. 4, though a controller 150 may include more or fewer elements than depicted in FIG. 1D or FIG. 4.

The first interface 156, the second interface 157, the third interface 158 and the fourth interface 159 may include ports, disk drives, wireless antennas, or other types of receiver circuitry. The first interface 156, the second interface 157, the third interface 158 and the fourth interface 159 connect the controller 150 to other components, devices and systems. For example, when the controller 150 is implemented in the central computer 110, the four interfaces may connect the central computer to any four or more of the main memory 115, the manager/provider computer 120, the first mobile computer 121 and the second mobile computer 122, the imaging system 170 and the display 180

The controller 150 may perform some of the operations described herein directly and may implement other operations described herein indirectly. For example, the controller 150 may indirectly control operations such as by generating and transmitting content to be displayed on the display 180. Accordingly, the processes implemented by the controller 150 when the processor 152 executes instructions from the memory 151 may include steps not directly performed by the controller 150.

FIG. 1E illustrates a system overview for dynamic medical supply procurement, in accordance with a representative embodiment.

The system overview in FIG. 1E includes five components including component 1, component 2, component 3, component 4 and component 5. The components may all be implemented by the central computer 110, or may be distributed among the central computer 110 and the manager/provider computer 120, and/or other elements shown in FIG. 1A. FIG. 1B and/or FIG. 1C. The components may also be implemented as and differentiated as different sets of software programs or sub-programs, including programs or sub-programs that may be executed in parallel such as by different cores of a multi-core processor. In FIG. 1E, component 1 is for data collection and preparation: component 2 is for supply insight generation to improve clinical outcomes: component 3 is for supply insight generation to improve operational and financial efficiency: component 4 is for real-time predicting to predict additional supplies during the procedure, and component 5 is for a user interface to communicate and request supplies.

In the system of FIG. 1E, data collection and preparation at 161 is component 1 and is based on data from databases, and feedback from the supply insight generation at 162 and the real-time predicting at 164. The data collection and preparation at 161 provides data to the databases, the supply insight generation at 162, the supply insight generation at 163 and the real-time predicting at 164. As an example, component 1 may capture the clinical information, operational information and workflow information from multiple information systems in a given hospital or network of hospitals. The data used in component 1 may originate from sources such as electronic medical records, radiology information systems and cardiology information systems. The various sources that provide data to component 1 may be highly diverse, with different data types, data models, formats, and semantics. Component 1 may be responsible for interacting with the different data sources to extract clinical and operational information associated with a clinical exam. The interfacing with clinical data sources, operational data sources and demographic data sources may be performed with any of a variety of available state-of-the-art information technology communication protocols such as HL7 (Health Level 7), DICOM (digital imaging and communications in medicine) and FHIR (fast healthcare interoperability resources).

As used herein, clinical information may refer to clinical concepts and patient demographic information that is usually associated with clinical documents such as clinical notes, radiology reports, and medical history. Example of clinical concepts and patient demographic information captured by component 1 include demographic information (e.g. age, gender, race, height, weight, etc.), smoke history, medical history, history of chest pain, allergies (e.g. contrast allergies, medication allergies), vital signs (height, weight, blood pressure, etc.) before the medical procedure, and chronical diseases (e.g. diabetes).

The clinical information used in component 1 may be further processed to capture clinical concepts present in the informatics healthcare systems. As examples of clinical concepts, smoke history, echocardiogram exam history, left ventricle (LV) history, enlarged LV history, cardiologists history, and ultrasound history may be captured from the clinical information used in component 1. Several ontologies may define a published list of clinical concepts that may used in component 1. Examples of ontologies which may be used to define clinical concepts include SNOMED CT (systemized nomenclature of medicine—clinical terms) and RadLex (lexicon of radiological information produced by the Radiological Society of North America). As examples, natural language processing (NLP) algorithms which use supervised and unsupervised approaches to identify clinical concepts in free-text reports may be used to capture clinical concepts. Another example is the use of RegEx (regular expression) expressions to query for clinical concepts in databases.

Operational information may refer to information relating to the operation of a given hospital or department(s) in a hospital. The operational information may be automatically captured from electronic systems or may be manually entered using a user interface. Examples of the information captured by component 1 include supplies utilized in the procedure (including supplies being used time): experience of healthcare providers, preferences of healthcare providers, use of contrast, length of procedures, staff involved in the procedures such as nurses, technicians, operators, and anesthesiologists, types of access, location of procedures, types of procedures, and protocols used for procedures.

The information captured in component 1, component 2, component 3, component 4 and component 5 in FIG. 1E may be stored in relational or non-relational databases.

During a medical procedure, detailed information gathered during the procedure may also be extracted, aggregated and linked with data obtained through component 1. The information gathered during the procedure may include, but is not limited to, a sub-procedure log including detailed steps of each sub-procedure/step and its time: identification of a supply used during the procedure: measurements taken during the procedure: vital signs measured during the procedure; and other information obtained during the procedure such as medical images. The information obtained during the procedure may be extracted from a CVIS (cardiovascular information system) and/or EMR (electronic medical records system). A time stamp for any of the information obtained during a medical procedure may also be extracted. In this way, the workflow and progression of a medical procedure may be captured and the information describing when the supply was used and at which stage may be mapped.

Supply insight generation at 162 is component 2 and is based on data from the data collection and preparation at 161. The supply insight generation at 162 is designed to improve clinical outcomes. The supply insight generation at 162 provides supply insights to the real-time predicting at 164 and to the user interface 165.

Component 2 may analyze clinical guideline adherence, such as for device usage and patient outcomes, to identify physician training opportunities for outcome improvement. Since the field of medical devices continually produces better and newer medical devices, patient clinical outcomes may be improved by using the most appropriate devices based on clinical evidence. Several ways of analyzing the clinically appropriate usage of devices may be used. One analysis method is through clinical guidelines and user-defined pathways. Another analysis method is through data-driven modeling. Use of clinical guidelines and user-defined pathways may use clinical guidelines published on when and how to use certain medical devices appropriately, based on clinical trial evidence of better clinical outcomes.

As an example of the processing involving component 2, use of a fractional flow reserve (FFR) device is suggested when there is intermediate or severe (<90%) stenosis, since use of the FFR device may better guide decisions in intervention medical procedures with improved patient outcomes. FFR is a measurement for determining a ratio between the maximum achievable blood flow in a diseased coronary and the theoretical maximum flow in a normal coronary artery. A user may also define pathways on when and how medical devices can be used in the hospital based on clinical guidelines and expert consensus. Data-driven modeling may be used to suggest the appropriate usage of devices, and is based on historical clinical outcomes associated with device usage. Machine learning or deep learning models may be trained to predict clinical outcomes using patient characteristics, surgery information, medical exams, diagnosis information, and device usage. Risk factors for adverse clinical outcomes may be included in the models to compare the device usage fairly. Component 2 may also be used in suggesting device usage.

Supply insight generation at 163 is component 3 and is based also on data from the data collection and preparation at 161. The supply insight generation at 163 is to improve operational and financial efficiency. The supply insight generation at 163 also provides supply insights to the real-time predicting at 164 and to the user interface 165.

Component 3 generates supply insight to improve operational and financial efficiency. Component 3 may use the information from component 1 to create insights about supply utilization, taking into consideration the financial aspects of facilities such as the catheterization laboratories. By providing an overview of supplies used in a given period of time, healthcare providers and managers may be provided an ability to understand supply utilization, pattern, trends, and cost reduction suggestions.

Component 3 may be relatively independent of other components, other than receiving input data from component 1 and component 2. Alternatively, component 3 may obtain similar input from databases such as inventory management systems. Component 3 may be implemented with sub-components, such as an insight generation algorithm, a user interface for managers, and a user interface for healthcare providers. The insight generation algorithm may receive input from component 1, such as supply usage, healthcare provider information, and procedure information, and this input may be integrated/linked together to perform further analysis. Billing or cost information may be used to analyze cost reductions. Matrix or key performance indicators may be used to reflect supply usage performance of providers. Different techniques may be used for insight generation, including time series analysis (for prediction/trending), machine learning, and deep learning. Root cause analysis and contributing factor analysis may also be applied.

An example of insight provided by the insight generation algorithm of component 3 is the creation of rules to favor more effective devices. That is, medical staff often become comfortable using some type of supplies, and may not be aware of new supplies that are less expensive and more effective. The insight generation algorithm of component 3 may identify supply usage patterns, and financial and clinical outcomes associated with the supply usage patterns, and may benchmark the supply usage patterns and financial and clinical outcomes with different user populations. The result may be suggestions for financial improvements, as well as the rationale involved in a final decision. Another example of insight provided by the insight generation algorithm is identification of usage outliers. Usage outliers may be identified based on the staff involved in a given procedure. Supply information may be used, taking into consideration the clinical information and operational information. For example, an elderly patient with history of heart failure and several comorbidities may require a higher level of care and supply than a younger patient. Based on the insights generated by the insight generation algorithm of component 3, management may suggest additional training to the medical staff.

The action suggestion sub-component of component 3 may suggest actions to take in order to improve supply usage and reduce costs after pain points of current supply utilization are identified by component 3. Discrete event simulation (DES) and/or machine learning/deep learning methods may be used to predict outcomes with potential improvement if a suggestion is adopted. Discrete event simulation is a method of simulating behavior and performance of an actual process, facility or system. DES models the process, facility of system as a series of ‘events’ that occur over time. In a health-care context, events may include births, stays in an intensive care unit (ICU), a transfer or a discharge, for example. Patients may be modelled as independent entities, each of which can be given associated attribute information such as age, weight, location, and the attribute information may be modified. The DES simulation also accounts for resources, such as requirements for cots, nursing staff and equipment. DES thus allows complex decision logic to be incorporated that is not as readily possible in other types of modelling. The DES simulation also allows scenario-testing, to enhance understanding alternative ways in which a new policy may be best met, and thus may be used to predict outcomes with potential improvements if a suggestion is adopted.

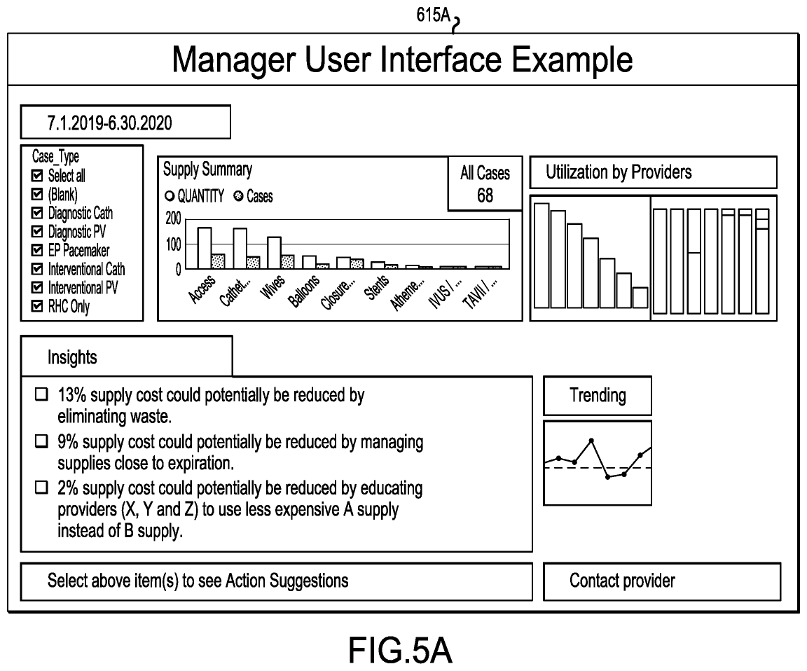

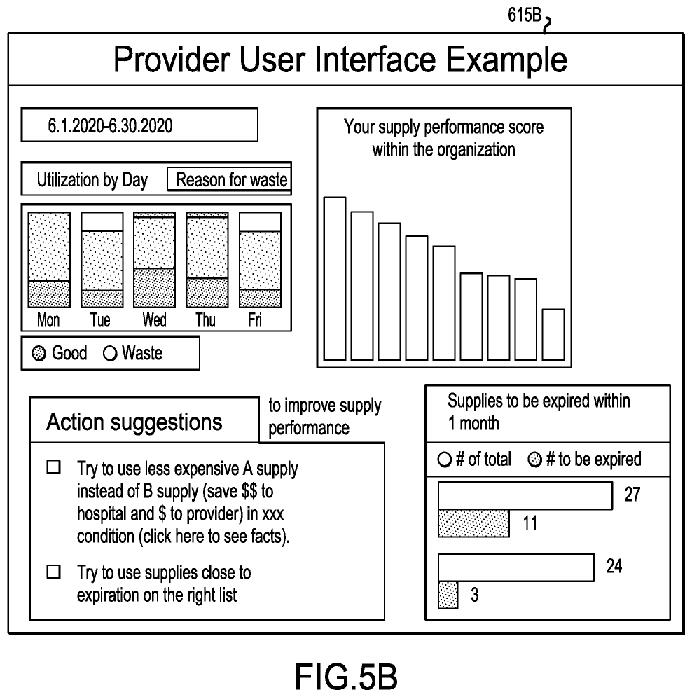

The user interface sub-component of component 3 may provide separate user interfaces for healthcare managers and healthcare providers. For managers, the healthcare manager user interfaces may provide an overview of supply utilization, a provider's performance on supply utilization, and insights from component 3. A dashboard may integrate sub-components to provide deep dive options. An example of a healthcare manager user interface is shown in FIG. 5A, and an example of a provider's user interface is shown in FIG. 5B.

Real-time predicting at 164 is component 4 and is based on the data from the data collection and preparation at 161, the supply insight generation at 162 and the supply insight generation at 163. The real-time predicting at 164 provides a real-time prediction as output to a user or automated ordering system, such as to order supplies for immediate delivery. The real-time predicting at 164 also provides the real-time predictions to the data collection and preparation at 161.

Component 4 provides an ability to predict additional supplies during a procedure. Information from component 1 may be used as the starting point to allow healthcare providers to create an initial setup of supplies that are used in a given procedure. Due to the dynamic and complex nature of operation rooms such as the catheterization laboratories, an extra supply that was not previously selected, but which is needed, may be identified during the progression of the medical procedure. Component 4 captures the clinical information and operational information in real-time during the medical procedure to predict the extra supply that should be obtained during a procedure. Component 4 may include three sub-components for intra-procedure data extraction and preparation, model training, and real-time predicting.

The model training may use time series with multi-channel feature information from component 1, deep learning algorithms, time series analysis or a combination of the algorithms and analysis to train a classification model with supply being used or not as the outcome. An example of a deep learning algorithm is a recurrent neural network (RNN). Due to the variations in frequency of data from different features, data interpolation methods may be applied before making predictions. As an example, vital signs may be collected more frequently than other types of data. Real-time predicting may involve applying a model trained with historical data, in real-time for new procedures. Both the probability of supply being used and a confidence level may be associated with each predicted supply so that healthcare providers may judge whether a supply should be requested and/or prepared before the procedure.

An example of use for component 4 in the catheterization laboratories is for diagnostic catheterization procedures. A physician may be provided an ability to decide whether to perform PCI (percutaneous coronary intervention) during the procedure after diagnosing with X-ray and possibly other modalities such as FFR or wire. The stenosis level may be measured and recorded, along with the FFR value. Accordingly, the possibility of using balloon and stent may increase compared to the prediction based only on pre-procedure information. As a result, the number of supplies needed may be predicted more confidently. The possibility of requiring more supplies may increase with collection of the PCI decision information. The supply usage possibility and confidence level may be continuously updated as additional information is collected during the procedure. If a new device is predicted to have a high probability of being used, nurses/physician may prepare the new device before the new device is needed to avoid idle waiting time, such as to prepare a 3rd stent of different size that would be needed in 10 minutes.

The user interface 165 is component 5 and is based on data from the databases, and the supply insight generation at 162 and the supply insight generation at 163. The user interface provides data to the databases.

Component 5 is responsible for using the information gathered in component 1, component 2, component 3 and component 4 to help healthcare managers organize and request supplies. Using the real-time model in component 4 to predict the supplies required during a give procedure, component 5 may automatically send requests to add more supplies that are newly needed in a timely manner. Knowing how much supplies will be used during a given procedure in real-time and requesting the supplies that may not otherwise be prepared may help improve workflow efficiency, to avoid rescheduling the procedure due to shortages of supplies and thus to improve clinical and financial outcomes.

As an example of use for component 5, a real-time supply prediction by component 4 may be based on newly acquired X-Ray images and IVUS images during a diagnostic catheterization procedure, and may suggest using three drug-eluting stents (DES), one of length 17 mm and two of length 15 mm. If the current catheterization laboratory only has two satisfactory DES prepared, another DES may be shipped to the catheterization laboratory quickly before the catheter is pulled out, so that the patient doesn't need to schedule a staged catheterization procedure for a third stent to be placed.

Another application of component 5 is to communicate with clinicians during a medical procedure to suggest the most appropriate supplies. Component 5 may identify the most appropriate supply with acceptable clinical outcomes, efficient workflow and reduced financial cost based on component 2, component 3 and component 4. Suggestions may be communicated through user interfaces with the clinicians who perform the operations to help with their supply decisions during the operations. For example, the central computer 110 may notify a clinician at the first mobile computer 121 or the second mobile computer 122 to suggest a replacement for the imaging system 170, or to simply suggest ordering additional quantities of another type of supply.

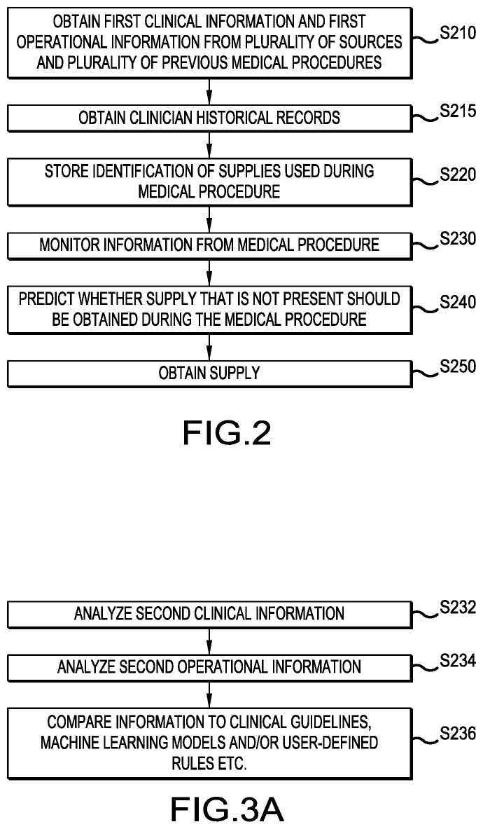

FIG. 2 illustrates a method for dynamic medical supply procurement, in accordance with a representative embodiment.

The method of FIG. 2 may be performed by the system 100A in FIG. 1A, the system 100B in FIG. 1B or the system 100C in FIG. 1C.

At S210, the method of FIG. 2 includes obtaining first clinical information and first operational information from a plurality of sources and a plurality of previous medical procedures. A central computer 110 may establish benchmarks from a plurality of previous medical procedures. Benchmarks may include types and amounts of supplies used for medical procedures involving specific characteristics.

The first clinical information may comprise many different types of information, along with results of correlating different types of information. As an example, usage of a type of device by a clinician may be determined from historical records of which types of devices are used by clinicians for specific types of medical procedures. A controller 150 of the central computer 110 may determine familiarity of a clinician present during the medical procedure with the supplies used during the medical procedure.

At S215, clinician historical records are obtained. For example, clinical historical records of a subject may be obtained from an EMR system or another type of system that stores historical clinical records of a subject of a medical procedure.

At S220, the method of FIG. 2 includes storing identification of supplies used during a medical procedure. S220 may be performed before the medical procedure, such as when the medical procedure is being planned. Accordingly, supplied used during a medical procedure may be identified before the medical procedure, and stored at S220.

At S230, the method of FIG. 2 includes monitoring information from the medical procedure. The monitoring at S230 may be performed directly by the central computer 110, and indirectly from information from medical systems such as the imaging system 170, information from mobile devices such as the first mobile computer 121 and the second mobile computer 122, and information from other sources such as sensors and monitors. The monitoring information may be provided to the central computer 110 for analysis to ensure the adequacy of the supplies identified for the medical procedure at S220.

When the information from the medical procedure is from the imaging system 170, the information from the medical procedure may be based on images of a subject of the medical procedure taken during the medical procedure and processed in real-time. A simple example of use of images of a subject is when the information shows poor contrast in the images, such that a replacement imaging system of the same type or of a different type should be obtained immediately. In this example, the supply that is not present during the medical procedure may be obtained based on a quality of the images of the subject, and the supply that is not present but which is obtained may comprise a replacement medical imaging apparatus.

At S240, the method of FIG. 2 includes predicting whether a supply that is not present should be obtained during the medical procedure. The predicting at S240 may be performed periodically or continually during the medical procedure while the information from the medical procedure is monitored at S230. The predicting at S240 may also be performed for any type of supply used during the medical procedure, for may be limited to only types of supplies designated to be of high importance for the medical procedure.

If S240 results in a prediction that a supply should be obtained, at S250 the method of FIG. 2 includes obtaining the supply. The supply may be obtained by alerting a staff member to immediately retrieve the supply and deliver the supply to the medical procedure, or may be obtained by generating a request for immediate delivery of the supply to an automated system.

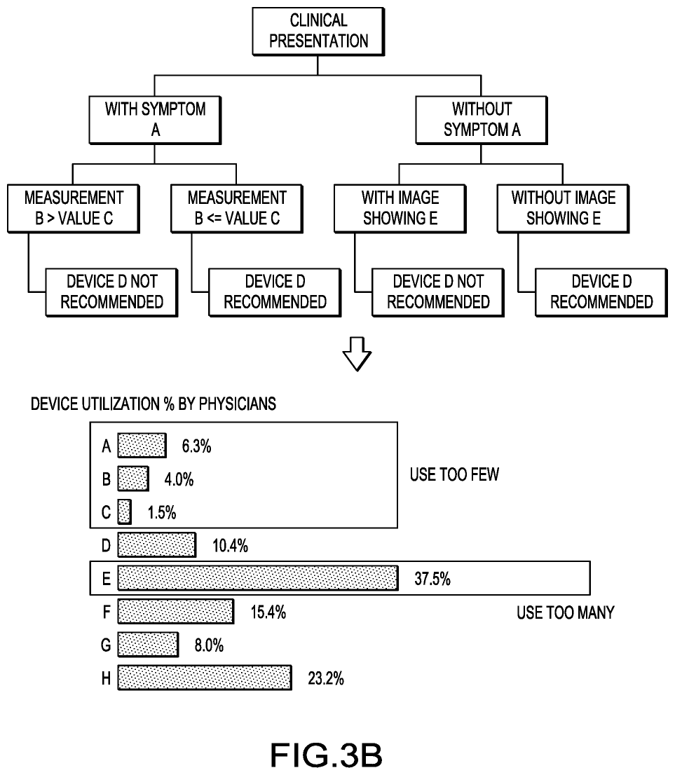

FIG. 3A illustrates a method for dynamic medical supply procurement, in accordance with a representative embodiment.

The method of FIG. 3A may be performed during or after S230 in FIG. 2.

At S232, the method of FIG. 3A includes analyzing second clinical information. The second clinical information may be from the medical procedure, such as clinical information generated during and based on the medical procedure. The second clinical information may be information from medical systems such as the imaging system 170, information from mobile devices such as the first mobile computer 121 and the second mobile computer 122, and information from other sources such as sensors and monitors.

At S234, the method of FIG. 3A includes analyzing second operation information. The second operational information may be from the medical procedure, such as clinical information generated during and based on the medical procedure. The analyzing at S234 may be performed by the central computer 110.

At S236, the method of FIG. 3A includes comparing the second clinical information from S232 and the second operational information from S234 to clinical guidelines, machine-learning models and/or user-defined rules. The comparing at S236 may be performed by the central computer 110. The comparing may be performed to determine when the medical procedure is outside of the clinical guidelines, when the machine-learning models indicate that a supply should be obtained for the medical procedure, or when the second clinical information indicates that the clinical procedure is violating the user-defined rules.

FIG. 3B illustrates insight generation for dynamic medical supply procurement, in accordance with a representative embodiment.

Insight generation for clinical outcome improvement is based on clinical guidelines. The insight generation may be performed by component 2 (i.e., the supply insight generation at 162) of FIG. 1E. As an example, in FIG. 3B the decision tree may reflect clinical guidelines for appropriate usages of a fractional flow reserve (FFR) device. The device utilization information shows the appropriate usage improvement opportunity for physicians. For example, physician A, B, C may increase FFR device usage according to clinical guidelines, and this may help improve patient outcomes, while physician E may decrease FFR device usage according to clinical guidelines.

In FIG. 3B, a clinical presentation is shown at the top level of a decision tree. The second level checks for the presence or absence of a symptom. On the left, the third level compares measurement values B with a threshold value C to determine whether a device D is recommended or not recommended at the fourth level. On the right, the third level determines whether a medical image shows characteristic E or not to determine whether the device D is recommended or not. In other words, interactive feedback from a medical procedure is compared to clinical guidelines while a medical procedure is occurring to determine whether a device D should be present. The interactive feedback may be information automatically collected and analyzed, such as medical images from the imaging system 170 analyzed by the central computer 110. The interactive feedback may also be information collected from staff present at the medical procedure, such as staff using the first mobile computer 121 or the second mobile computer 122.

As an example of FIG. 3B, the clinical presentation shown at the top is fed to a decision tree. For example, a first decision from the clinical presentation may be whether the medical procedure involves a stable angina or an unstable angina. If the first decision results in determination that an angina is stable, a second determination may be whether a legion is intermediate or severe. If the first decision results in a determination that an angina is unstable, a second determination may be whether a lesion is a culprit or not a culprit. Clinical guidelines may be applied at a 3rd level or 4th level to demonstrate whether an FFR is recommended or is not recommended. When the FFR is recommended, the result is compared to clinical guidelines for whether the FFR is less than or equal to 0.80, and the result in turn is used to determine whether to apply a percutaneous coronary intervention (PCI) or an optical medical therapy (OMT).

The insight generation in FIG. 3B is representative of a process that may be performed for numerous supplies for any particular medical procedure. The insight generation in FIG. 3B may also be performed repeatedly for a single supply for any particular medical procedure, such as periodically or each time an event triggers the insight generation.

FIG. 4 illustrates a computer system, on which a method for dynamic medical supply procurement is implemented, in accordance with another representative embodiment.

Referring to FIG. 4, the computer system 400 includes a set of software instructions that can be executed to cause the computer system 400 to perform any of the methods or computer-based functions disclosed herein. The computer system 400 may operate as a standalone device or may be connected, for example, using a network 401, to other computer systems or peripheral devices. In embodiments, a computer system 400 performs logical processing based on digital signals received via an analog-to-digital converter.

In a networked deployment, the computer system 400 operates in the capacity of a server or as a client user computer in a server-client user network environment, or as a peer computer system in a peer-to-peer (or distributed) network environment. The computer system 400 can also be implemented as or incorporated into various devices, such as the central computer 110, the first mobile computer 121, the second mobile computer 122, the manager/provider computer 120, or any other machine capable of executing a set of software instructions (sequential or otherwise) that specify actions to be taken by that machine. The computer system 400 can be incorporated as or in a device that in turn is in an integrated system that includes additional devices. In an embodiment, the computer system 400 can be implemented using electronic devices that provide voice, video or data communication. Further, while the computer system 400 is illustrated in the singular, the term “system” shall also be taken to include any collection of systems or sub-systems that individually or jointly execute a set, or multiple sets, of software instructions to perform one or more computer functions.

As illustrated in FIG. 4, the computer system 400 includes a processor 410. The processor 410 may be considered a representative example of the processor 152 of the controller 150 in FIG. 1D and executes instructions to implement some or all aspects of methods and processes described herein. The processor 410 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 410 is an article of manufacture and/or a machine component. The processor 410 is configured to execute software instructions to perform functions as described in the various embodiments herein. The processor 410 may be a general-purpose processor or may be part of an application specific integrated circuit (ASIC). The processor 410 may also be a microprocessor, a microcomputer, a processor chip, a controller, a microcontroller, a digital signal processor (DSP), a state machine, or a programmable logic device. The processor 410 may also be a logical circuit, including a programmable gate array (PGA), such as a field programmable gate array (FPGA), or another type of circuit that includes discrete gate and/or transistor logic. The processor 410 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 400 further includes a main memory 420 and a static memory 430, where memories in the computer system 400 communicate with each other and the processor 410 via a bus 408. Either or both of the main memory 420 and the static memory 430 may be considered representative examples of the memory 151 of the controller 150 in FIG. 1B, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 420 and the static memory 430 are articles of manufacture and/or machine components. The main memory 420 and the static memory 430 are computer-readable mediums from which data and executable software instructions can be read by a computer (e.g., the processor 410). Each of the main memory 420 and the static memory 430 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 400 further includes a image/video display unit 450, such as a liquid crystal display (LCD), an organic light emitting diode (OLED), a flat panel display, a solid-state display, or a cathode ray tube (CRT), for example. Additionally, the computer system 400 includes an input device 460, such as a keyboard/virtual keyboard or touch-sensitive input screen or speech input with speech recognition, and a cursor control device 470, such as a mouse or touch-sensitive input screen or pad. The computer system 400 also optionally includes a disk drive unit 480, a signal generation device 490, such as a speaker or remote control, and/or a network interface device 440.

In an embodiment, as depicted in FIG. 4, the disk drive unit 480 includes a computer-readable medium 482 in which one or more sets of software instructions 484 (software) are embedded. The sets of software instructions 484 are read from the computer-readable medium 482 to be executed by the processor 410. Further, the software instructions 484, when executed by the processor 410, perform one or more steps of the methods and processes as described herein. In an embodiment, the software instructions 484 reside all or in part within the main memory 420, the static memory 430 and/or the processor 410 during execution by the computer system 400. Further, the computer-readable medium 482 may include software instructions 484 or receive and execute software instructions 484 responsive to a propagated signal, so that a device connected to a network 401 communicates voice, video or data over the network 401. The software instructions 484 may be transmitted or received over the network 401 via the network interface device 440.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

FIG. 5A illustrates a user interface for a manager/provider computer for dynamic medical supply procurement, in accordance with a representative embodiment.

In FIG. 5A, a user interface 615A is for a manager. The user interface 615A may be provided via the manager/provider computer 120. The user interface 615A presents information for a period of time from Jul. 1, 2019 to Jun. 30, 2020 as an example. The information includes a selection of case type, a supply summary for 68 cases showing quantity bars on the left versus a cases bar on the right for each of a set of supplies. The information also includes use of different supplies by providers, trends, and insights. The insights in FIG. 615A show a manager the potential savings in supply costs if waster were to be eliminated, the potential savings in supply costs if supplies were managed close to expiration, and the potential savings in supply costs if specific individual providers (staff members) were to be educated to use a less expensive supply A instead of a more expensive supply B.

FIG. 5B illustrates another user interface for a manager/provider computer for dynamic medical supply procurement, in accordance with a representative embodiment

In FIG. 5B, a user interface 615B is for a provider. The user interface 615B may be provided via the manager/provider computer 120. The user interface 615B presents information for a period of time from Jul. 1, 2020 to Jun. 30, 2020 as an example. The information includes a selection of use of a supply by day, and percentages of usage or wastage of the supply for each day. The user interface 615B also shows a comparison of the provider's supply performance score within the provider's organization, so comparative with the provider's peers. The user interface 615B also shows action suggestions, such as switching to a less expensive supply, and/or using supplies close to expiration. The user interface 615B also shows supplied to be expired within 1 month, including relative numbers of such supplies compared to total numbers.

Accordingly, dynamic medical supply procurement enables customization of supply provisioning in real-time in accordance with staff preferences and abilities, and patient conditions. The real-time prediction of supply requirements during procedures may be supported by medical data such as medical images and physiology information from the procedures. Dynamic medical supply procurement may be implemented in electronic systems for hospitals or departments, such as a hospital information system or cardiovascular information system.

Although dynamic medical supply procurement has been described with reference to several exemplary embodiments, it is understood that the words that have been used are words of description and illustration, rather than words of limitation. Changes may be made within the purview of the appended claims, as presently stated and as amended, without departing from the scope and spirit of dynamic medical supply procurement in its aspects. Although dynamic medical supply procurement has been described with reference to particular means, materials and embodiments, dynamic medical supply procurement is not intended to be limited to the particulars disclosed: rather dynamic medical supply procurement extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method for dynamically obtaining a supply, the method comprising:
storing, in a main memory, identification of supplies used during a medical procedure;
monitoring information from the medical procedure during the medical procedure;
predicting, by a processor executing instructions and based on monitoring the information, whether a supply that is not present should be obtained during the medical procedure, and
generating an alert indicating the supply that is not present during the medical procedure based on predicting that the supply that is not present should be obtained.

2. The method of claim 1, further comprising:
analyzing clinical information input to a communication device and received from the communication device at a computer to determine compliance with clinical guidelines during the medical procedure.

3. The method of claim 1, further comprising:
analyzing operational information input to a communication device and received from the communication device at a computer to determine usage of supplies during the medical procedure.

4. A system for dynamically obtaining a supply, the system comprising:
a central computer comprising a first memory that stores first instructions and a first processor that executes the first instructions;
a main memory that stores identification of supplies used during a medical procedure;
wherein, when executed by the first memory, the first instructions cause the central computer to:
monitor information from the medical procedure during the medical procedure;
predict, based on monitoring the information, whether a supply that is not present should be obtained during the medical procedure, and
obtain the supply that is not present during the medical procedure based on predicting that the supply that is not present should be obtained.

5. The system of claim 4, wherein, when executed by the first processor, the first instructions cause the central computer further to:
before the medical procedure, capture first data from a plurality of sources to obtain first clinical information and first operational information used to determine the supplies used during the medical procedure;
during the medical procedure, monitor the information from the medical procedure to obtain second clinical information and second operational information to predict whether the supply that is not present should be obtained during the medical procedure.

6. The system of claim 4, wherein, when executed by the first processor, the first instructions cause the central computer further to:
compare the information from the medical procedure to clinical guidelines;
determine, based on comparing the information from the medical procedure to the clinical guidelines, whether the supplies used during the medical procedure comply with the clinical guidelines; and
obtain the supply that is not present during the medical procedure further based on determining that the supplies used during the medical procedure do not comply with the clinical guidelines.

7. The system of claim 4, further comprising:
a mobile computer comprising a second memory that stores second instructions and a second processor that executes the second instructions, wherein, when executed by the second processor, the second instructions cause the mobile computer to:
obtain the information from the medical procedure during the medical procedure, and send the information from the medical procedure to the central computer.

8. The system of claim 4, wherein, when executed by the first processor, the first instructions cause the central computer further to:
obtain information from previous medical procedures; and
establishing benchmarks from a plurality of the previous medical procedures,
wherein, whether a supply that is not present should be obtained during the medical procedure is predicted based further on the benchmarks from the plurality of the previous medical procedures.

9. The system of claim 4, wherein, when executed by the first processor, the first instructions cause the central computer further to:
automatically generate and send a request for the supply that is not present to obtain the supply that is not present.

10. The system of claim 4, wherein the information from the medical procedure is based on images of a subject of the medical procedure taken during the medical procedure and processed in real-time.

11. The system of claim 4, wherein the information from the medical procedure is obtained from a plurality of different sources over an electronic communication network.

12. A controller, comprising:
a memory that stores instructions; and
a processor that executes the instructions, wherein, when executed by the processor, the instructions cause the controller to:
obtain identification of supplies used during a medical procedure;
monitor information from the medical procedure during the medical procedure;
predict, based on monitoring the information, whether a supply that is not present should be obtained during the medical procedure, and
generating an alert indicating the supply that is not present during the medical procedure based on predicting that the supply that is not present should be obtained.

13. The controller of claim 12, wherein, when executed by the processor, the instructions cause the controller further to:
obtain images of a subject of the medical procedure taken during the medical procedure and processed in real-time; and
obtain the supply that is not present during the medical procedure based on a quality of the images of the subject, wherein the supply that is not present comprises a medical imaging apparatus.

14. The controller of claim 12, wherein, when executed by the processor, the instructions cause the controller further to:
determine familiarity of a clinician present during the medical procedure with the supplies used during the medical procedure; and
obtain the supply that is not present during the medical procedure based on the familiarity of the clinician present during the medical procedure with the supplies used during the medical procedure.

15. The controller of claim 14, wherein, when executed by the processor, the instructions cause the controller further to:
interpret the information from the medical procedure using natural language processing.
